(19) United States
(12) Patent Application Publication
(10) Pub. No.: US 2003/0054422 A1
(43) Pub. Date: Mar. 20, 2003

(54) Lipopolysaccharide immunoassay and test device

(76) Inventors: Robert Andrew Badley, Bedford (GB); Glen Hughes, Macclesfield (GB); Krzsztof Wojciech Zak, Bedford (GB)

Correspondence Address: OPPEDAHL AND LARSON LLP, P O BOX 5068, DILLON, CO, 80435-5068, US

(21) Appl. No.: 10/131,433

(22) Filed: Apr. 23, 2002

Related U.S. Application Data
(62) Division of application No. 09/545,180, filed on Apr. 7, 2000.

(30) Foreign Application Priority Data
Apr. 7, 1999 (EP) 99302711.9

(51) Int. Cl. G01N 33/554; G01N 33/569

(52) U.S. Cl. 435/7.32

(57) ABSTRACT

An immunoassay for detecting lipopolysaccharides, and a device for conducting the same, the immunoassay comprising the steps of:

a) contacting the sample with a first binding reagent selected from the group consisting of an antibody having specific binding affinity to the lipopolysaccharide analyte and a lipopolysaccharide binding protein, wherein the sample and first binding reagent are brought into contact for a sufficient time to allow for the lipopolysaccharide analyte, if any, to bind to the first binding reagent to form a first binding reagent/lipopolysaccharide analyte complex;

b) contacting the first binding reagent/lipopolysaccharide analyte complex with a second binding reagent selected from the group consisting of an antibody having specific binding affinity to the lipopolysaccharide analyte and a lipopolysaccharide binding protein, wherein the first binding reagent/lipopolysaccharide analyte complex and the second binding reagent are brought into contact for a sufficient time to allow for the first binding reagent lipopolysaccharide analyte complex to bind to the second binding reagent to form a first and second binding reagent/lipopolysaccharide analyte complex; and

c) detecting the presence of first and second binding reagent/lipopolysaccharide analyte complex formed;

wherein one of the binding reagents is an antibody having specific binding affinity to the lipopolysaccharide analyte, and the other binding reagent is a lipopolysaccharide binding protein; and further

wherein one of the binding reagents a labelled binding reagent, preferably one which is capable of migrating to the location of the other reagent, which is unlabelled and preferably immobilized on a solid support.

### FIELD OF THE INVENTION

This invention relates to immunoassays involving specific binding, and to test devices for carrying out such assays. In particular, this invention relates to immunoassays (and test devices) involving the use of both monoclonal or polyclonal antibodies and proteins capable of binding an identified analyte.

### BACKGROUND OF THE INVENTION

Immunoassays have been described which make use of the specific binding properties of antibody-type molecules to detect the presence of an analyte of interest in a sample. The antibody-type molecules suitable for such assays may be whole antibodies—monoclonal or polyclonal—or they may be antibody fragments, e.g. Fv and Fab fragments.

To be of practical use in immunoassays, the specific binding affinity of antibody-type molecules to analytes of interest is not, in and of itself, adequate. The immunoassays must also provide a means by which measurement of the level of binding of the antibody-type molecule and analyte can be measured. In this regard, it has generally become accepted for the antibody-type molecule to be linked to a label of some sort. Known labels include radioactive, fluorescent, electroactive (such as redox labels) or chemiluminescent compounds, enzymes, and particulate labels (such as latex beads or gold sol).

Basically, two general forms of immunoassays are known and have been constructed utilising the aforementioned components. Competition assays, in a general nature, detect the presence of a particular analyte in a sample by providing an environment whereby the analyte can cause the displacement either of a labelled antibody-type molecule or ligand from a pre-existing complex, the result being that relatively high levels of analyte in a sample yield low signals and relatively low levels of analyte yield high signals. Various forms of competition assays are described in detail in Schuurs et al. U.S. Pat. No. 3,654,090, and Badley et al. WO 97/44664.

The other general form of immunoassay is often termed the sandwich-type assay. Typically in such assays a complex is built up, either sequentially or simultaneously, consisting of an antigen with at least two antibodies of the same or different specificities bound to it. Frequently, one of the antibodies is attached to a solid surface and one will have attached to it some form of label for detection of the sandwich complex. Because of their structure, such sandwich-type assays provide for analyte signals which are directly proportional to the amount of analyte in the sample. Various forms of sandwich assays are described in detail in Davis et al. EP 0042755 and P. Tijssen Practice and Theory of Enzymatic Immunoassays, published by Elsevier (Amsterdam, 1985).

Both forms of immunoassays described above may be utilised to identify the presence of a wide range of analytes. Hormonal analytes such as human chorionic gonadotrophin, lutenizing hormone or estrone, or their metabolic biproducts, may be identified from urine, serum or other bodily fluids, as is described May et al. U.S. Pat. No. 5,656,503. Other potential analytes include cancer markers, pesticides and metabolites.

Certain analytes which are markers for pathogenic species are also desirable to assay as a means to identify infection at an early enough stage so as to be able to provide timely and effective treatment. In this regard, it has been known to assay for bacteria and viruses. Representative assays for the detection of these and other analytes are described in Rapid Methods and Automation in Microbiology and Immunology, Ed. Spencer et al., published by Intercept (United Kingdom, 1994).

One such analyte, which serves as a marker for pathogenic species, is lipopolysaccharide. Lipopolysaccharides (LPS) are an essential component of all Gram-negative bacterial outer membranes and are thought to be the principal agents responsible for inflammatory responses in patients infected by such bacteria. Gram-negative bacteria include Escherichia coli, and species of Salmonella (e.g. S. minnesota and S. typhimurium), Chlamydia (e.g. C. trachomatis) and Neisseria (e.g. N. gonorrhoeae and N. lactamica).

The structure of LPS has been determined as consisting of three distinct regions: a lipid A region, a core oligosaccharide, and an O-polysaccharide chain consisting of long chains of repeating oligosaccharide units. LPS having all three of these regions is referred to as a smooth or S-form chemotype. It is also known, though, that certain mutants of Gram-negative bacteria, as well as certain naturally occurring Gram-negative bacteria, produce what is known as a rough or R-form chemotype, a form of LPS having no O-polysaccharide region. Moreover, within the R-form chemotype, additional variants are known to exist. For example, certain mutants of Eschericia coli and Salmonella, and naturally occuring Chlamydia, produce LPS having only a partial core oligosaccharide.

Since the structure of the LPS varies so considerably among bacterial species, it has been thought desirable to design species-specific immunoassays through the use of antibodies having a specific binding affinity for the species-specific LPS in question. Pronovost et al. WO 97/06436, describes one such immunoassay. Specifically, it discloses a sandwich-type immunoassay for detecting LPS which utilizes two distinct antibodies capable of binding to the LPS.

It is known, however, that most antibodies to LPS have a specific binding affinity for the variable core oligosaccharide region of the bacteria. Because of this, immunoassays utilizing two distinct antibodies may have limited efficacy as the antibodies may compete for the same epitope on LPS or, because of the close proximity of epitopes, the antibodies may hinder each other's respective binding reaction.

One possibility for eliminating the foregoing problems is to provide two antibody immunoassays wherein one of the antibodies binds to the lipid A region of LPS. However, few antibodies have been identified which are capable of specific binding to fatty acids, and none have been shown to bind directly to the lipid A region of LPS derived from Gram-negative bacteria. Thus, there has been only limited success in providing effective immunoassays for detecting the presence of Gram-negative bacteria, especially sandwich-type assays which require multiple binding events to occur with respect to each analyte or antigen to be detected.

It has been known, though, to provide other means for binding a second component to LPS in a sandwich-type assay. Mertsola et al., Detection of Experimental Haemophilus influenza Type b Bacteremia and Endotoxemia by Means of an Immunolimulus Assay, April 1991, The Journal of Infectious Diseases, vol. 164 pp. 353-358 draws upon prior work done by Levin and Bang (The Role of Endotoxin in the Extracellular Coagulation of Limulus Blood, April 1964, Bulletin of Johns Hopkins Hospital vol. 115, pp. 265-274) and others with respect to the Limulus amebocyte lysate (LAL) system. It discloses an immunoassay method whereby LAL, a complex lysate, is used in combination with unlabelled monoclonal antibodies to detect Haemophilus influenza type b. In such an assay, the antibodies are immobilised on a microtiter plate. LPS is captured by the antibodies through formation of a complex with the LPS molecule's core oligosaccharide region. Detection of the bound LPS then occurs by activation of the LAL system.

The mechanism by which the LAL system operates is not entirely understood, but it is thought that signal generation in LPS assays containing LAL is the result of a cascade of enzymatic reactions, initiated by the complexing of a protein with the LPS and resulting in activation of an enzyme capable of converting a chromogenic substrate to a chromophore, thus providing a detectable signal.

The immunoassay described by Mertsola et al differs from most existing commercially marketed assays for the detection of LPS in that a second antibody is not required to generate a detectable signal. However, it suffers from the disadvantage of utilising a complex extract, the LAL itself. Such an extract, as it is derived from living organisms with their inherent differences, can give rise to assays that have unacceptable variability. Furthermore, as LAL requires an enzymatic cascade to occur for the generation of a detectable signal, any LAL containing assay will necessarily proceed at a relatively slow rate, which may be undesirable in certain commercial settings.

### SUMMARY OF THE INVENTION

It would therefore be desirable to provide an immunoassay capable of overcoming the foregoing disadvantages, and to provide an analytical test device capable of performing such an immunoassay. In this regard, the present invention provides a method of detecting the presence of a lipopolysaccharide analyte in a sample, the method comprising the steps of:

a) contacting the sample with a first binding reagent selected from the group consisting of an antibody having specific binding affinity to the lipopolysaccharide analyte and a lipopolysaccharide binding protein, wherein the sample and first binding reagent are brought into contact for a sufficient time to allow for the lipopolysaccharide analyte, if any, to bind to the first binding reagent to form a first binding reagent/lipopolysaccharide analyte complex;

b) contacting the first binding reagent/lipopolysaccharide analyte complex with a second binding reagent selected from the group consisting of an antibody having specific binding affinity to the lipopolysaccharide analyte and a lipopolysaccharide binding protein, wherein the first binding reagent/lipopolysaccharide analyte complex and the second binding reagent are brought into contact for a sufficient time to allow for the first binding reagent lipopolysaccharide analyte complex to bind to the second binding reagent to form a first and second binding reagent/lipopolysaccharide analyte complex; and

c) detecting the presence of first and second binding reagent/lipopolysaccharide analyte complex formed;

wherein one of the binding reagents is an antibody having specific binding affinity to the lipopolysaccharide analyte, and the other binding reagent is a lipopolysaccharide binding protein; and further

wherein one of the binding reagents is a labelled binding reagent, preferably one which is capable of migrating to the location of the other binding reagent, which is unlabelled and preferably immobilised on a solid support.

Also contemplated is an analytical test device for performing such an assay. The device comprises a solid support having reversibly immobilised thereon in a first zone of the support a labelled binding reagent selected from the group consisting of an antibody having specific binding affinity to the lipopolysaccharide analyte and a lipopolysaccharide binding protein, the solid support further having irreversibly immobilised thereon in a second zone of the support an unlabelled binding reagent selected from the group consisting of an antibody having specific binding affinity to the lipopolysaccharide analyte and a lipopolysaccharide binding protein, the second zone being a detection zone for the presence of the analyte in the sample.

In the device only one of the binding reagents should be an antibody having specific binding affinity to the lipopolysaccharide analyte, the other being a lipopolysaccharide binding protein. Further, the solid support should be such as to be capable upon contact with a liquid biological sample of conveying by capillary action the sample and the unlabelled binding reagent into the detection zone.

The immunoassay of the invention provides for assay devices to be constructed which are capable of accurate and reproducible test results, ideally suited for commercial markets such as the clinical or home-testing markets. Furthermore, such immunoassays can be performed quickly and simply, with little need for the use of complex extracts or multiple assay steps. Such simplicity is again ideal for such commercial settings as the clinical or home-testing markets.

### DETAILED DESCRIPTION OF THE INVENTION

The immunoassays of the present invention are utilised to identify the presence or absence of lipopolysaccharide analytes (LPS) in a given sample. Thus, the assays of the invention are capable of identifying whether a sample contains Gram-negative bacteria, such as Escherichia coli, Salmonella, or Chlamydia, which have incorporated into their cell membranes species-specific variants of LPS.

As the LPS to be detected in the sample is an integral component of the Gram-negative bacteria's cell membrane, it is contemplated that prior to application of the principle assay steps to the sample (that is, prior to contacting the sample with the first binding reagent, as discussed below), extraction of the LPS from the cell membrane preferably occur. Extraction may be performed by any known means. Preferably, zwitterionic detergents such as 3-([chloramidopropyl]-dimethylamino)-1-propanesulfonate (CHAPS) or non-ionic detergents such as polyethyleneglycol octyphenyl ether will be applied to the sample at levels sufficient to cause lysing of the bacterial cell membrane, but below levels at which inhibition of the assay will occur. Most typically, these levels will be between about 0.01% (wt/vol) and about 2.0% (wt/vol), though greater and lesser levels are specifically contemplated.

The immunoassays may be applied to virtually any type of sample, though liquid biological samples derived from urine, serum, saliva, cervical or urethral fluids, and stool samples are most likely to contain the bacteria for which the assays are concerned. Other liquids which are suitable for the assays of the invention include food samples and samples from surfaces. The samples may be purified or diluted prior to assaying.

Once extracted from the cell membrane of its host bacteria, LPS is capable of being assayed in accordance with the methods of the invention. Specifically, the methods of the invention are directed to “sandwich-type” immunoassays, examples of which are described in Davis et al. EP 0042755 and P. Tijssen, Practice and Theory of Enzymatic Immunoassays, published by Elsevier (Amsterdam, 1985). Such sandwich-type assays require that multiple binding events occur with respect to any LPS in a given sample. In this regard, it is contemplated that in the inventive assay, LPS will first be brought into contact with, and will bind to, a first binding reagent selected from either an antibody having specific binding affinity to the LPS, or a lipopolysaccharide binding protein, to form a first binding reagent/LPS complex. The resulting first binding reagent/LPS complex is then contacted with a second binding reagent selected from either an antibody having specific binding affinity to the LPS, or a lipopolysaccharide binding protein, to form a second complex which is capable of providing a detectable signal to the tester.

It is contemplated that any conventional method may be used to provide contact between LPS and the first binding reagent, and between the first binding reagent/LPS complex and the second binding reagent. Exemplary methods range from capillary action, which occurs in conventional strip (eg nitrocellulose) containing test devices, to simple diffusion of one or more of the components in a solution to the location of the other components, which occurs in certain forms of ELISA-type immunoassays and Energy Transfer Immunoassays.

If the first binding reagent is an antibody, it is preferred that the antibody be a monoclonal antibody, though it is specifically contemplated that polyclonal antibodies may also be used. Antibodies may additionally include any functional antibody fragments such as Fab, Fv, and even smaller units such as heavy chain variable fragments, which may be prepared by biochemical or antibody engineering methods from known whole antibodies or indirectly from libraries of antibodies or antibody-like molecules (see, for example, Verhoeyen and Windust, Advances in Antibody Engineering in Molecular Immunology: Frontiers in Molecular Biology, 2nd Ed., published by Oxford University Press, pp. 283-325 (Oxford, 1995)). It is significant that the antibody exhibit a specific binding affinity to the LPS of interest; that is, that the antibody bind to the LPS of interest in a selective fashion in the presence of excess quantities of other materials including other LPS molecules not of interest, and tightly enough (with high enough affinity) to provide a useful assay. For example, the antibody might bind only to Chlamydia LPS or particular chemotypes of S. typhimurium, but not to other chemotypes.

Polyclonal antibodies and monoclonal antibodies may be prepared by any suitable known procedure, including those described in Price et al, Principles and Practice of Immunoassays, 2nd Ed., published by Macmillan Publishers Ltd (London, 1997).

Monoclonal Antibodies specific for LPS, as well as their methods of manufacture are also discussed in Poxton, Antibodies to Lipopolysaccharide, May 1995, Journal of Immunological Methods, vol. 185, pp. 1-15 and Luk et al, Epitope Mapping of Four Monoclonal Antibodies Recognizing the Hexose Core Domain of Salmonella Lipopolysaccharide, December 1991, Journal of Biological Chemistry.

The first binding reagent may instead be a lipopolysaccharide binding protein. Such proteins may be single or multi-chain polypeptides; they may be simple or conjugated; and/or they may be fibrous or globular. They will, however, be other than an antibody or portion thereof (for example, an Fv or Fab sequence). Further, they will exhibit a binding affinity to the LPS of interest, which means they will have a general specificity to most if not all chemotypes of LPS in the presence of excess quantities of other materials.

As the immunoassay of the invention is a sandwich assay, the lipopolysaccharide binding proteins will have a binding affinity to a site on the LPS molecule which is different from the site which is bound, in the second step of the assay, by the antibody. This will ensure that both first and second binding reagents are each capable of binding to the LPS molecule. Most typically, the proteins will bind to the lipid portion of the LPS molecule. Thus, they are not species-specific, and will bind to any LPS derived from any Gram-negative bacteria. For this reason, in order to provide an immunoassay for the detection of LPS derived from a particular species, it is necessary to include as binding reagents both a lipopolysaccharide binding protein and an antibody having specific binding affinity to a particular species dependent form of LPS.

The lipopolysaccharide binding proteins can be prepared by known conventional methods. Proteins such as those described in Wright et al., A Receptor for Complexes of Lipopolysaccharide (LPS) and LPS Binding Protein, 1990, Science vol. 249, pp. 1431-1433; Bazil et al. Biochemical Characterization of a Soluble Form of the 53-kDa Monocyte Surface Antigen, 1986, Eur.J.Immunol vol. 16(12), pp. 1583-1589; Lehrer et al Antibacterial Activity of Microbicidal Cationic Proteins 1 and 2, Natural Peptide Antibiotics of Rabbit Lung Macrophages, 1983, Infect. Immun. vol. 42(1), pp. 10-14; Juan et al., Identification of a domain in soluble CD14 essential for LPS signalling but not LPS binding, 1995, J.Biol.Chem. vol. 270(29), pp. 17237-17242; Haas et al. A Synthetic Lipopolysaccharide-Binding Peptide, 1998, J.Immunol. vol. 161, pp. 3607-3615; and Selsted et al., Primary Structures of Six Antimicrobial Peptides of Rabbit Peritoneal Neutrophils, 1985, J.Biol.Chem., vol. 260(8), pp. 4579-4584, are suitable for use in the invention, along with lipopolysaccharide binding proteins isolated from the Limulus amebocyte lysate (LAL) described in Mertsola et al. Detection of Experimental Haemophilus influenza Type b Bacteremia and Endotoxemia by Means of an Immunolimulus Assay, April 1991, The Journal of Infectious Diseases, vol. 164, pp. 353-358. Purification of LAL lipopolysaccharide binding proteins from LAL may be accomplished by methods known in the art. For example, such proteins may be obtained by heparin-Sepharose chromatography, as described by Aketagawa J et al., Primary Structure of Limulus Anticoagulant Anti-lipopolysaccharide Factor, 1986, J.Biol.Chem. vol. 261(16), pp. 7357-7365.

In a preferred embodiment of the invention, the lipopolysaccharide binding protein is polypeptide, more particularly a single chain polypeptide having a molecular weight of less than about 5000 Da, as measured by laser desorption (MALDI) mass spectroscopy. More preferably, it is a single chain polypeptide having a molecular weight of between 3000 and 4000 Da, as measured in the same manner. Specifically suitable polypeptides have been sequenced by selective proteolytic cleavage methods. In particular, it is preferred to utilise a polypeptide comprising an amino acid sequence which is at least 90%, more preferably at least 95% and optimally 100%, identical to an amino acid sequence selected from

SEQ ID NO:1:

Gly Leu Arg Lys Arg Leu Arg Lys Phe Arg Asn Lys
Ile Lys Glu Lys Leu Lys Lys Ile Gly Gln Lys Ile
Gln Gly Leu Leu Pro Lys Leu Ala

SEQ ID NO:2:

His Glu Cys His Tyr Arg Ile Lys Pro Thr Phe Arg
Arg Leu Lys Trp Lys Tyr Lys Gly Lys Phe Trp Cys
Pro Ser

SEQ ID NO:3:

Asp His Glu Cys His Tyr Arg Ile Lys Pro Thr Phe
Arg Arg Leu Lys Trp Lys Tyr Lys Gly Lys Phe Trp
Cys Pro Ser

SEQ ID NO:4:

Asn Gln Gly Arg His Phe Cys Gly Gly Ala Leu Ile
His Ala Arg Phe Val Met Thr Ala Ala Ser Cys Phe
Gln

SEQ ID NO:5:

Asn Ala Asn Cys Lys Ile Ser Gly Lys Trp Lys Ala
Gln Lys Arg Phe Leu Lys Met Ser Gly Asn Phe Asp
Cys Ser Ile

SEQ ID NO:6:

Asp Ser Ser Ile Arg Val Gln Gly Arg Trp Lys Val
Arg Lys Ser Phe Phe Lys Leu Gln Gly Gly Ser Phe
Asp Val Ser Val

Subtantially longer or shorter polypeptide sequences such as those described below by SEQ ID NO:7 and SEQ ID NO:8, respectively, are also specifically contemplated.

SEQ ID NO:7:

Thr Thr Pro Glu Pro Cys Glu Leu Asp Asp Glu Asp
Phe Arg Cys Val Cys Asn Phe Ser Glu Pro Gln Pro
Asp Trp Ser Glu Ala Phe Gln Cys Val Ser Ala Val
Glu Val Glu Ile His Ala Gly Gly Leu Asn Leu Gly
Pro Phe Leu Lys Arg Val Ala Asp Ala Asp Pro

SEQ ID NO:8:

Glu Lys Pro Leu Gln Asn Phe Thr Leu Cys Phe Arg
Ala

It is most preferred for the immunoassays of the present invention to incorporate a polypeptide comprising the amino acid sequence of SEQ ID NO:1.

Having contacted the first binding reagent (whether it be an antibody having specific binding affinity to LPS or a lipopolysaccharide binding protein) with LPS in the sample, a first binding reagent/lipopolysaccharide analyte complex forms. This complex is then brought into contact with a second binding reagent to form a second complex, the first and second binding reagent/lipopolysaccharide analyte complex. It is intended that like the first binding reagent, the second binding reagent also be selected from either an antibody having specific binding affinity to the LPS or a lipopolysaccharide binding protein. However, the present invention requires that of the two binding reagents, only one may be an antibody having specific binding affinity to LPS, the other binding reagent necessarily being a lipopolysaccharide binding protein. Thus, the invention contemplates two distinct alternative scenarios, namely that (a) the first binding reagent is an antibody and the second binding reagent is a lipopolysaccharide binding protein; or (b) the first binding reagent is a lipopolysaccharide binding protein and the second binding reagent is an antibody.

If an antibody is utilised as the second binding reagent, then those antibodies suitable for use, as well as the preferred ones, are as described above with respect to the first binding reagent. Likewise, if the second binding reagent is a lipopolysaccharide binding protein, it is also as described above with respect to the first binding reagent.

In the preferred embodiment of the present invention, one of the binding reagents will be irreversibly immobilised on a solid support (e.g. a “well” surface as described for ELISA-type assays in Davis et al. EP 0042755, or a nitrocellulose strip as described in May et al., U.S. Pat. No. 5,656,503) and the other binding reagent will be capable of migrating to the location of the immobilised one. Furthermore, the binding reagent which is characterised by being capable of migrating to the immobilised binding reagent will be conjugated with a label of some sort, so as to provide a means by which to detect the presence of the first and second binding reagent/lipopolysaccharide analyte complex. The irreversibly immobilized binding reagent will be unlabelled.

The label that is conjugated to the migrating binding reagent can be any entity the presence of which can be readily detected. Preferably the label is a direct label, such as the those described in detail in May et al., U.S. Pat. No. 5,656,503. Direct labels are entities which, in their natural state, are readily visible either to the naked eye, or with, the aid of an optical filter and/or applied stimulation, e.g. UV light to promote fluorescence. Examples include radioactive, chemiluminescent, electroactive (such as redox labels), and fluorescent compounds. Direct particulate labels, such as dye sols, metallic sols (e.g. gold), non-metallic elemental particles, such as carbon particles and selenium particles, and coloured latex particles, are also very suitable and are preferred. Of these options, coloured latex particles are most preferred. Concentration of the label into a small zone or volume should give rise to a readily detectable signal, e.g. a strongly coloured area. This can be evaluated by eye, or by instruments if desired.

Indirect labels, such as enzymes, e.g. alkaline phosphatase and horseradish peroxidase, can also be used, but these usually require the addition of one or more developing reagents such as substrates before a visible signal can be detected. Hence, they are less preferred. Such additional reagents can be incorporated in the solid support such that they dissolve or disperse when a liquid sample is applied. Alternatively, the developing reagents can be added to the sample before application of the sample to the solid support.

Conjugation of the label to the migrating binding reagent can be by covalent or non-covalent (including hydrophobic) bonding, if desired, or by adsorption. It may also be accomplished by coupling through an avidin/biotin “bridge”. Techniques for such conjugation are commonplace in the art and may be readily adapted for the particular binding reagents and labels utilised in the present invention. In the preferred embodiment, the label is a coloured latex particle and it is conjugated to the migrating binding reagent through adsorption.

The analytical test device capable of performing the immunoassays of the invention may take any one of numerous forms, depending on the precise nature of the assay being performed (e.g. the analyte of interest, the type of support, the particular binding reagents etc.). In a preferred embodiment, it is contemplated that the device comprise a solid support which has reversibly immobilised thereon in a first zone a labelled binding reagent selected from either an antibody having specific binding affinity to the lipopolysaccharide analyte or a lipopolysaccharide binding protein, the solid support further having irreversibly immobilised thereon in a second zone of the support an unlabelled binding reagent selected from either an antibody having specific binding affinity to the lipopolysaccharide analyte or a lipopolysaccharide binding protein, the second zone being a detection zone for the presence of the analyte in the sample; wherein only one of the binding reagents is an antibody having specific binding affinity to the lipopolysaccharide analyte, the other being a lipopolysaccharide binding protein, and further wherein the solid support is characterised in that it is capable upon contact with a liquid biological sample of conveying by capillary action the sample and the unlabelled binding reagent into the detection zone.

Such devices can be of the general type described in, for example, May et al., U.S. Pat. No. 5,622,871 and May et al., U.S. Pat. No. 5,656,503. Preferably, these devices comprise a hollow elongated casing containing the solid support, which most typically is a dry porous carrier. The solid support communicates indirectly with the exterior of the casing via a bibulous fluid sample receiving member which may or may not protrude from the casing, the solid support and the sample receiving member being linked so as to allow for the fluid sample to migrate between the two by capillary action, the solid support having a first zone wherein a binding reagent (preferably a lipopolysaccharide binding protein) bearing a label is reversibly immobilised—that is, the binding reagent is immobilised on the dry solid support but becomes freely mobile on or within the solid support when the support becomes moist. Such reversible immobilisation of the binding reagent in the first zone may be accomplished in any one of a number of known ways (e.g. as described in, for example, Taylor Protein Immobilisation, published by Marcel Dekker, Inc., 1991). Specifically, it is preferred that such immobilisation occur by adsorption to the support, as described in May et al., U.S. Pat. No. 5,622,871.

Spatially distant along the solid support from the sample receiving member is a second zone (the detection zone) on which an unlabelled and different binding reagent (preferably an unlabelled antibody having specific binding affinity to LPS) is irreversibly immobilised. By “irreversibly immobilised” it is meant that the unlabelled binding reagent will be incorporated on or bound to the solid support in such a way as to prevent its migration when it (or the support) is moist. Irreversible immobilisation of the binding reagent to the solid support may be accomplished in any one of a number of ways, each of which will be apparent to the person skilled in the art. The binding reagent may be chemically coupled to the support using, for example, CNBr, carbonyldiimidazole, or tresyl chloride, or by avidin/biotin coupling. Alternatively, various “printing” techniques may be used. These include application of liquid binding reagents by micro-syringes, direct printing, ink-jet printing, and the like. Chemical or physical treatment of the support prior to application of the binding reagent is also specifically contemplated, as such may facilitate immobilisation.

The device may be constructed as described above with respect to the preferred embodiment, or it may be constructed wherein the reversibly immobilised binding reagent is a labelled antibody and the unlabelled irreversibly immobilised binding reagent is a lipopolysaccharide binding protein.

The casing in the preferred devices is typically constructed of opaque or translucent material incorporating at least one aperture through which the analytical result may be observed, preferably visibly observed by the naked eye.

Such devices can be provided to clinical laboratories or as kits suitable for home use, such kits comprising one or more devices individually wrapped in moisture impervious wrapping and packaged together with appropriate instructions to the user.

The sample receiving member can be made from any bibulous, porous or fibrous material capable of absorbing liquid rapidly. The porosity of the material can be unidirectional (i.e. with pores or fibres running wholly or predominantly parallel to an axis of the member) or multidirectional (omnidirectional, so that the member has an amorphous sponge-like structure). Porous plastics material, such as polypropylene, polyethylene (preferably of very high molecular weight), polyvinylidene fluoride, ethylene vinylacetate, acrylonitrile and polytetrafluoro-ethylene can be used. It can be advantageous to pre-treat the member with a surface-active agent during manufacture, as this can reduce any inherent hydrophobicity in the member and therefore enhance its ability to take up and deliver a moist sample rapidly and efficiently. Porous sample receiving members can also be made from paper or other cellulosic materials, such as nitrocellulose. Preferably the material comprising the sample receiving member should be chosen such that the porous member can be saturated with liquid sample within a matter of seconds. The liquid must be capable of permeating freely from the porous sample receiving member into the solid support.

The solid support is most preferably a dry porous carrier. It may be made of several strips arranged, for example, in series, or separate strips or sheets and, like the sample receiving member, it can be constructed from any material or combination of materials capable of allowing the liquid sample to migrate through a portion of its length by, preferably, capillary action. The support should allow for the immobilisation of the binding reagents on its surface, and should not interfere with the binding reactions which form the binding reagent/lipopolysaccharide analyte complexes. Preferably, the solid supports are made from paper, nylon, nitrocellulose or any surface having suitable flow characteristics. Most preferred are those made from nitrocellulose.

The solid support may have associated with it an absorbent “sink” which will facilitate capillary action of fluid up the length of the support. Specific materials for and applications of sinks are conventional in the art and may be readily applied to the devices of the present invention.

The invention can be better appreciated by reference to the following specific examples. They are intended to be illustrative and not exhaustive of the methods and devices of the invention.

### EXAMPLES

These examples demonstrate how Chlamydia LPS can be detected via an analytical test device which employs a labelled lipopolysaccharide binding protein and, on a solid support, an irreversibly immobilised and unlabelled antibody having specific binding affinity to the LPS.

Preparation of Antibody and Lipopolysaccharide Binding Protein:

The monoclonal antibody (Mab7) used as a binding reagent in the following experiment was prepared according to procedures known in the art. A representative method for generating monoclonal antibodies is described by Gani et al J. Steroid Biochem. Molec. Biol. 1994, vol. 48, pp. 277-282) and this method can be adapted to produce a relevant antibody having specific binding affinity to LPS derived from Chlamydia. A suitable monoclonal antibody can be selected on the basis of its relative affinity and specificity for the analyte and analyte analogues. This can be undertaken, for example, by performing standard kinetic determinations using a Biacore™ 2000 biosensor (Biacore AB, Sweden), as described in the manufacturer's protocol (Applications handbook, Biacore AB), using a panel of closely related analogues.

The lipopolysaccharide binding protein utilised in this example was provided by Affinity Research Products, United Kingdom. Specifically, the lipopolysaccharide binding protein is a polypeptide derived from a known polypeptide (CAP18) isolated from rabbit granulocytes. The protein can be isolated from perotoneal granulocytes as described by Hitata et al., Infection & Immunity, 1994, vol. 62, pp. 1421-1426, and the peptide sequence determined by sequencing methods disclosed in Larrick et al., Biochem Biophys Res. Commun. vol. 179, pp. 170-175. Specifically, the peptide (Peptide 1465) had a molecular weight of 3801.4 Da, as measured by laser desorption (MALDI) mass spectroscopy, and comprised the amino acid sequence represented by SEQ ID NO.1.

Conjugation of a Label to Peptide 1465:

Conventional particulate labels comprising coloured (blue) polystyrene latex particles (0.5% wt/vol latex solids, diameter approx. 400 nm) were obtained from Duke Laboratories, Durham N.C. USA. Peptide 1465 was then adsorbed on to the surface of the latex particles at a concentration of 100 μg/ml by mixing in phosphate buffered saline for 30 min at 46° C.

Deposition of Mab7 onto a Solid Support:

Mab7 was deposited on a solid support comprising a nitrocellulose strip, the Mab7 being irreversibly immobilised in a detection zone on the strip.

Specifically, nitrocellulose strips 340 mm long by 40 mm wide were coated in a detection zone (10 mm from the bottom edge) with Mab7, at a level of 1.2 mg/ml, at a plotting rate of 0.1 μl/mm using an automated X-Y plotter. The plotted strips were dried at 55° C. The strips were then treated with 1% (wt/vol) polyvinyl alcohol containing 3% (wt/vol) sucrose, and air dried at a temperature of 70° C. For Examples 1 and 2, strips 6 mm wide were used.

### Example 1

In this example, purified Chlamydia-like lipopolysaccharide derived from S. typhimurium (Re595) was shown to be detectable by the above-described analytical test device.

Re595 LPS was obtained from Sigma Chemical Co., Poole, United Kingdom. Multiple dilutions in accordance with Table 1 were prepared. Specifically, Re595 LPS was diluted to 100 μg/ml in phosphate buffered saline. The Re595 LPS dilution was further diluted in phosphate buffered saline by serial 10 fold dilutions to 1 pg/ml.

TABLE 1
Dilutions of Re595 LPS

| Dilution | [LPS]/ml | |
|---|---|---|
| 1 | 100 | μg |
| 2 | 10 | μg |
| 3 | 1 | μg |
| 4 | 100 | ng |
| 5 | 10 | ng |
| 6 | 1 | g |
| 7 | 100 | pg |
| 8 | 10 | pg |
| 9 | 1 | pg |

| | |
|---|---|
| 10 | Neg |
| | control |

Ten microlitres of Peptide 1465 sensitised latex were then added to 1 ml of each of the Re595 LPS dilutions, mixed thoroughly, and 180 μl of each dilution was applied by pipette to one end of a nitrocellulose test strip containing the irreversibly immobilized Mab7. The dilution was allowed to migrate via capillary action into the detection zone containing the irreversibly immobilised Mab7. Line development (i.e. detection of Re595 LPS) was visually monitored and measured by scanning densitometry 5 minutes after sample application.

The results of this Example are shown in Table 2. It demonstrates that the latex particle labelled Peptide 1465 was capable of capturing Re595 LPS so as to form, in conjunction with the irreversibly immobilised Mab7 antibody, a visible blue line in the detection zone on the nitrocellulose test strip. Assay signal is taken as an increase in blue line density at the detection zone. It can be seen in Table 2 that dilutions 1-4, though still demonstrating a significant assay signal, nevertheless showed lower assay signal than certain subsequent dilutions. It is believed that this is due to latex particle aggregation at the base of the strips due to higher Re595 LPS concentrations.

TABLE 2
Assay Signal
Results for Re595 LPS with Peptide 1465

| LPS | Assays |
|---|---|
| Dilution | signal |
| 1 | 6.95 |
| 2 | 6.63 |
| 3 | 12.7 |
| 4 | 18.36 |
| 5 | 25.97 |
| 6 | 25.89 |
| 7 | 31.73 |
| 8 | 11.14 |
| 9 | 3.64 |
| control | 3.4 |

### Example 2

In this example, LPS incorporated within Chlamydia cell lysate was shown to be detectable by the above-described analytical test device.

Chlamydia trachomatis (LGV-2) was grown under routine tissue culture conditions in McCoy cells. The cell culture procedures followed were as described in Diagnostic Procedures for Viral, Rickettsial and Chlamydial Infections, 5th Ed. published by American Public Health Association, Inc., (Washington D.C.). Cells containing Chlamydia elementary bodies were harvested by scraping infected cells will a cell scraper from the monolayers, and treatment with formaldehyde solution at a final concentration of 1:1000 for 16 hours at 2-8° C. Cells were stored in a conventional freezer at −80° C. until required.

Prior to use, the Chlamydia cell lysate was allowed to thaw completely. The Chlamydia cell lysate was then diluted with phosphate buffered saline in a manner similar to that described in Example 1, except that the lysate was diluted 1:1626 in phosphate buffered saline. The resulting dilution was further diluted in phosphate buffered saline by serial two-fold dilutions as set forth in Table 3.

TABLE 3
Dilutions of Chlamydia lysate

| Dilution | Chlamydial |
|---|---|
| ID | dilution |
| 1 | 1 |
| 2 | 1/2 |
| 3 | 1/4 |
| 4 | 1/8 |
| 5 | 1/16 |
| 6 | 1/32 |
| 7 | 1/64 |
| 8 | neg |
| | control |

Ten microliters of Peptide 1465 sensitised latex were then added to 1 ml of each of the Chlamydia cell lysate dilutions, mixed thoroughly, and 180 μl of each dilution was applied by pipette to one end of a nitrocellulose test strip containing the irreversibly immobilised Mab7. The dilution was allowed to migrate via capillary action into the detection zone containing the irreversibly immobilised Mab7. Line development (i.e. detection of Chlamydia LPS in the lysate) was visually monitored and measured by scanning densitometry 5 minutes after sample application.

The results of this Example 2 are shown in Table 4 and are similar to those obtained in Example 1. The results demonstrate that the latex particle labelled Peptide 1465 was capable of capturing LPS found in Chlamydia cell lysate so as to form, in conjunction with the irreversibly immobilised Mab7 antibody, a visible blue line in the detection zone on the nitrocellulose test strip. Assay signal is taken as an increase in blue line density at the detection zone.

TABLE 4
Assay Signal
Results for Chlamydia cell lysate with Peptide 1465

| Chlamydial | |
|---|---|
| cell lysate | Assay |
| dilution | signal |
| 1 | 5.12 |
| 2 | 4.56 |
| 3 | 3.06 |
| 4 | 3.35 |
| 5 | 2.8 |
| 6 | 2.8 |
| 7 | 1.73 |
| Control | 1.98 |

While the invention has been described in detail and with respect to specific embodiments thereof, it will be apparent to one skilled in the art that various changes and modifications can be made therein without departing from the spirit and scope thereof.

What is claimed is:

1) A method of detecting the presence of a lipopolysaccharide analyte in a sample, the method comprising the steps of:
a) contacting the sample with a first binding reagent selected from the group consisting of an antibody having specific binding affinity to the lipopolysaccharide analyte and a lipopolysaccharide binding protein, wherein the sample and first binding reagent are brought into contact for a sufficient time to allow for the lipopolysaccharide analyte, if any, to bind to the first binding reagent to form a first binding reagent/lipopolysaccharide analyte complex;
b) contacting the first binding reagent/lipopolysaccharide analyte complex with a second binding reagent selected from the group consisting of an antibody having specific binding affinity to the lipopolysaccharide analyte and a lipopolysaccharide binding protein, wherein the first binding reagent/lipopolysaccharide analyte complex and the second binding reagent are brought into contact for a sufficient time to allow for the first binding reagent lipopolysaccharide analyte complex to bind to the second binding reagent to form a first and second binding reagent/lipopolysaccharide analyte complex; and
c) detecting the presence of first and second binding reagent/lipopolysaccharide analyte complex formed;
wherein one of the binding reagents is an antibody having specific binding affinity to the lipopolysaccharide analyte, and the other binding reagent is a lipopolysaccharide binding protein; and further
wherein one of the binding reagents is a labelled binding reagent and the other binding reagent is an unlabelled binding reagent.

2) A method according to claim 1 wherein one of the binding reagents is immobilised on a solid support and the other is capable of migrating to the location of the immobilised binding reagent, the migrating binding reagent characterised in that it is the labelled binding reagent.

3) A method according to claim 2 wherein the first binding reagent is a lipopolysaccharide binding protein, and further wherein the second binding reagent is an antibody having specific binding affinity to the lipopolysaccharide analyte and is immobilised on the solid support.

4) A method according to any of the preceding claims wherein the lipopolysaccharide analyte in the sample is derived from the cell membrane of a Gram-negative bacterium.

5) A method according to claim 4 wherein the lipopolysaccharide analyte in the sample is derived from the cell membrane of a Gram-negative bacteria selected from the group consisting of Escherichia coli, Salmonella and Chlamydia.

6) A method according to claim 5 wherein the lipopolysaccharide analyte in the sample is derived from the cell membrane of Chlamydia.

7) A method according to claim 4 wherein the sample is selected from the group consisting of urine, serum, saliva, cervical or urethral fluid, and wherein the method further comprises the step of contacting the sample with a sufficient amount of detergent to cause lysing of the Gram-negative bacteria cell membranes, this step occurring prior to contacting the sample with the first binding reagent.

8) A method according to any of the preceding claims wherein the labelled binding reagent comprises a particulate label.

9) A method according to any of the preceding claims wherein one of the binding reagents comprises an anti-Chlamydia lipopolysaccharide antibody.

10) A method according to any of the preceding claims wherein one of the binding reagents comprises a lipopolysaccharide binding protein that is a polypeptide having a molecular weight of less than about 5000 Da.

11) A method according to claim 10 wherein the polypeptide has a molecular weight of between 3000 and 4000 Da.

12) A method according to claim 11 wherein the polypeptide comprises an amino acid sequence which is at least 90% identical to an amino acid sequence selected from the group consisting of SEQ ID NO:1, SEQ ID NO:2, SEQ ID NO:3, SEQ ID NO:4, SEQ ID NO:5, and SEQ ID NO:6.

13) A method according to claim 12 wherein the polypeptide comprises an amino acid sequence which is selected from the group consisting of SEQ ID NO:1, SEQ ID NO:2, SEQ ID NO:3, SEQ ID NO:4, SEQ ID NO:5, and SEQ ID NO:6.

14) A method according to claim 13 wherein the polypeptide comprises an amino acid sequence of SEQ ID NO:1.

15) An analytical test device for detecting the presence of a lipopolysaccharide analyte in a liquid biological sample, the device comprising: a solid support having reversibly immobilised thereon in a first zone of the support a labelled binding reagent selected from the group consisting of an antibody having specific binding affinity to the lipopolysaccharide analyte and a lipopolysaccharide binding protein, the solid support further having irreversibly immobilised thereon in a second zone of the support an unlabelled binding reagent selected from the group consisting of an antibody having specific binding affinity to the lipopolysaccharide analyte and a lipopolysaccharide binding protein, the second zone being a detection zone for the presence of the analyte in the sample; wherein only one of the binding reagents is an antibody having specific binding affinity to the lipopolysaccharide analyte, the other being a lipopolysaccharide binding protein, and further wherein the solid support is characterised in that it is capable upon contact with the liquid biological sample of conveying by capillary action the sample and the unlabelled binding reagent into the detection zone.

16) A device according to claim 15 wherein the labelled binding reagent is an lipopolysaccharide binding protein.

17) A device according to claim 16 wherein lipopolysaccharide binding protein is polypeptide having a molecular weight of less than about 5000 Da.

18) A device according to claim 17 wherein the polypeptide has a molecular weight of between 3000 and 4000 Da.

19) A device according to claim 18 wherein the polypeptide comprises an amino acid sequence which is at least 90% identical to an amino acid sequence selected from the group consisting of SEQ ID NO:1, SEQ ID NO:2, SEQ ID NO:3, SEQ ID NO:4, SEQ ID NO:5, and SEQ ID NO:6, and the antibody is an anti-Chlamydia lipopolysaccharide antibody.

20) A device according to claim 19 wherein the polypeptide comprises an amino acid sequence which is selected from the group consisting of SEQ ID NO:1, SEQ ID NO:2, SEQ ID NO:3, SEQ ID NO:4, SEQ ID NO:5, and SEQ ID NO:6.

21) A device according to claim 20 wherein the polypeptide comprises an amino acid sequence of SEQ ID NO:1.

22) A device according to any of claims 15-21 wherein the labelled binding reagent comprises a particulate label.

23) A device according to any of claims 15-22 wherein the solid support is comprised of a dry porous carrier material.

24) A device according to any of claim 23 wherein the solid support is comprised of a nitrocellulose.

25) A device according to any of claims 15-24 wherein the liquid biological sample comprises fluid selected from the group consisting of urine, serum, saliva, cervical or urethreal fluid, and wherein lipopolysaccharide analyte is derived from the cell membrane of a Gram negative bacteria.

26) A device according to claim 25 wherein, the lipopolysaccharide analyte is derived from the cell membrane of a Gram-negative bacteria selected from the group consisting of Escherichia coli, Salmonella and Chlamydia.

27) A device according to claim 26 wherein the lipopolysaccharide analyte is derived from the cell membrane of Chlamydia.


SEQUENCE LISTING (8 sequences)

SEQ ID NO: 1 - protein, 32 aa, Lapine
GLRKRLRKFRNKIKEKLKKIGQKIQGLLPKLA

SEQ ID NO: 2 - protein, 26 aa, Limulus sp.
HECHYRIKPTFRRLKWKYKGKFWCPS

SEQ ID NO: 3 - protein, 27 aa, Limulus sp.
DHECHYRIKPTFRRLKWKYKGKFWCPS

SEQ ID NO: 4 - protein, 25 aa, Homo sapiens
NQGRHFCGGALIHARFVMTAASCFQ

SEQ ID NO: 5 - protein, 27 aa, Homo sapiens
NANCKISGKWKAQKRFLKMSGNFDCSI

SEQ ID NO: 6 - protein, 28 aa, Homo sapiens
DSSIRVQGRWKVRKSFFKLQGGSFDVSV

SEQ ID NO: 7 - protein, 59 aa, Lapine
TTPEPCELDDEDFRCVCNFSEPQPDWSEAFQCVSAVEVEIHAGGLNLGPFLKRVADADP

SEQ ID NO: 8 - protein, 13 aa, Lapine
EKPLQNFTLCFRA